(19) United States
(12) Patent Application Publication
(10) Pub. No.: US 2026/0144513 A1
(43) Pub. Date: May 28, 2026

(54) PROACTIVE DETECTION OF A COMPROMISED ATTACHMENT OF A GANTRY TO A FLOOR AND/OR A COMPONENT TO A ROTATING FRAME OF AN IMAGING SYSTEM

(71) Applicant: GE Precision Healthcare LLC, Waukesha, WI (US)

(72) Inventors: Brandon Allan Smith, Waukesha, WI (US); Joseph Daniel Maule, Menomonee Falls, WI (US); Ariel Friedlander, Mequon, WI (US)

(73) Assignee: GE Precision Healthcare LLC, Waukesha, WI (US)

(21) Appl. No.: 18/961,750

(22) Filed: Nov. 27, 2024

(51) Int. Cl. A61B 6/58; A61B 6/00; A61B 6/03; A61B 6/46

(52) U.S. Cl. CPC A61B 6/586; A61B 6/035; A61B 6/4435; A61B 6/461; A61B 6/547; A61B 6/582

(57) ABSTRACT

A computed tomography imaging system includes a gantry mounted to a floor. The gantry includes a rotating frame rotatably supported in the gantry, at least one component mounted to the rotating frame, and a gantry motion sensing system configured to sense, while the rotating frame is rotating, a motion of the gantry that is indicative of a state of one of an attachment of the gantry to the floor and an attachment of the at least one component to the rotating frame and generate a motion signal indicative of the state. Motion signal processing circuitry is configured to process the motion signal, and, in response to determining that the motion signal exceeds at least one predetermined threshold, transmit a notification that indicates at least one of the attachment of the gantry to the floor and the attachment of the at least one component to the rotating frame is compromised.

### FIELD

The following generally relates to computed tomography (CT), and more particularly to proactive detection of a compromised attachment of a gantry of a CT imaging system to an examination room floor and/or a compromised attachment of a component to a rotating frame of the CT imaging system, and is amenable to other systems that are attached to supports and include rotating components.

### BACKGROUND

A computed tomography (CT) imaging system generally includes a gantry that houses components utilized in the generation, emission and detection of X-rays. For example, the gantry houses a rotating frame that is rotatably supported via a bearing or the like in the gantry. The rotating frame includes an annular ring and is configured to rotate around a bore of the gantry along an axis of rotation about a center of the bore (i.e., an isocenter). The rotating frame carries components such as an X-ray source, a high voltage generation system, a data acquisition system, cooling system, balancing weights, etc.

During scanning, the rotating frame rotates around an object supported in the bore, the X-ray source emits X-ray radiation that traverses the object, and the detector array detects X-ray radiation traversing the subject object impinging the detector array. The detector array generates projection data (line integrals) indicative of the detected X-ray radiation. A reconstructor reconstructs the projection data and generates volumetric image data. Voxels of the volumetric image data are displayed as a two-dimensional (2-D) and/or a three-dimensional (3-D) image using gray scale values corresponding to a relative radiodensity.

In image space, the isocenter has been utilized as a center of the reconstructed image. Motion of the isocenter outside of manufacturer's specifications during scanning a patient can manifest as artifact, e.g., blur, etc., in the reconstructed image, reducing diagnostic image quality. During installation, the system undergoes testing to verify the imaging system is installed and operating in accordance with the manufacturer's specifications, including verifying that the gantry is properly attached to the examination room floor and that components are properly attached to the rotating frame.

In one example, the gantry is mounted to the examination room floor through nuts and bolts. For this, threaded gantry anchor bolts are embedded at predetermined locations in the examination room floor. A base of the gantry includes corresponding mounting hole locations. The gantry is positioned on the examination room floor such that the threaded gantry anchor bolts extend up through the mounting hole locations in the base. Anchor nuts are installed on the threaded gantry anchor bolts to secure the gantry to the examination room floor. Over time, the attachment can degrade, which can lead to additional motion of the gantry and hence the isocenter, negatively affecting image quality.

For example, examination room floor characteristics such as flatness, rigidness, etc. can become less flat, rigid, etc. over time. This may result in a compromised attachment of the gantry to the examination room floor. In another example, an anchor nut can loosen, an anchor bolt may detach from the examination room floor, etc. In another example, the base, a weld, a support, etc. associated with the attachment can become compromised. In such instances, gantry motion may fall out of specification, resulting in increased motion of the isocenter. Other sources of motion include vibration from nearby construction, other operating equipment, etc.

In another instance, an attachment of a component to the rotating frame can become compromised. For example, a nut securing a component to the rotating frame can loosen. In this instance, the component may become displaced from its mounted position, creating an imbalance or vibration in the rotating frame, resulting in increased motion of the isocenter. In another instance, the nut may “unscrew” from a bolt, e.g., due to vibration, forces, etc., with the nut and/or the bolt becoming loose in the gantry. Loose objects of the rotating frame can cause damage to the imaging system, e.g., where a loose object strikes another component, etc.

A compromised attachment of the gantry to the floor and/or a compromised attachment of a component to the rotating frame have been reactively detected, i.e., after degradation of image quality, damage to the imaging system, etc. during an inspection of the imaging system as a result of detection of the degradation of the image quality, the damage to the imaging system, etc. Unfortunately, this may result in additional X-ray radiation dose exposure to the patient where the patient is rescanned to meet image quality specifications, imaging system down-time for repairs and additional expenses for the repairs, etc.

In view of at least the foregoing, there is an unresolved need for an improved approach for detecting compromised attachments such as a compromised attachment of the gantry to the floor, a compromised attachment of a component to the rotating frame, etc. with systems such as a CT imaging system that includes attachments to supports and rotating components.

### SUMMARY

Aspects described herein address the above-referenced problems and others. This summary introduces concepts that are described in more detail in the detailed description. It should not be used to identify essential features of the claimed subject matter, nor to limit the scope of the claimed subject matter.

In one aspect, a computed tomography imaging system includes a gantry mounted to a floor. The gantry includes a rotating frame rotatably supported in the gantry, at least one component mounted to the rotating frame, and a gantry motion sensing system configured to sense, while the rotating frame is rotating, a motion of the gantry that is indicative of a state of one of an attachment of the gantry to the floor and an attachment of the at least one component to the rotating frame and generate a motion signal indicative of the state. Motion signal processing circuitry is configured to process the motion signal, and, in response to determining that the motion signal exceeds at least one predetermined threshold, transmit a notification that indicates at least one of the attachment of the gantry to the floor and the attachment of the at least one component to the rotating frame is compromised.

In another aspect, a computer-implemented method includes rotating a rotating frame of a gantry of a computed tomography imaging system, wherein the gantry is mounted to a floor and at least one component is mounted to the rotating frame, sensing a motion of the gantry that is indicative of a state of one of an attachment of the gantry to the floor and an attachment of the at least one component to the rotating frame and generate a motion signal indicative of the state, and transmitting, in response to determining that the motion signal exceeds at least one predetermined threshold, a notification that indicates at least one of the attachment of the gantry to the floor and the attachment of the at least one component to the rotating frame is compromised.

In another aspect, a computer readable medium is encoded with computer executable instructions. The computer executable instructions, when executed by a processor, cause the processor to: rotate a rotating frame of a gantry of a computed tomography imaging system, wherein the gantry is mounted to a floor and at least one component is mounted to the rotating frame, sense a motion of the gantry that is indicative of a state of one of an attachment of the gantry to the floor and an attachment of the at least one component to the rotating frame and generate a motion signal indicative of the state, and transmit, in response to determining that the motion signal exceeds at least one predetermined threshold, a notification that indicates at least one of the attachment of the gantry to the floor and the attachment of the at least one component to the rotating frame is compromised.

Those skilled in the art will recognize still other aspects of the present application upon reading and understanding the attached description.

### BRIEF DESCRIPTION OF THE DRAWINGS

The application is illustrated by way of example and not limited by the figures of the accompanying drawings in which like references indicate similar elements.

FIG. 1 schematically illustrates a non-limiting example of an imaging system configured for computed tomography imaging and including a gantry mounted to an examination room floor, a rotating frame with various components attached thereto, a gantry motion sensing system, and a gantry motion evaluation module, in accordance with an embodiment(s) herein.

FIG. 2 schematically illustrates a non-limiting example of a base portion of the gantry attached to the examination room floor via a support bracket, in accordance with an embodiment(s) herein.

FIG. 3 schematically illustrates a non-limiting example of the rotating frame with a plurality of components attached thereto, in accordance with an embodiment(s) herein.

FIG. 4 schematically illustrates a non-limiting example of a suitable location of the gantry motion sensing system in the gantry, in accordance with an embodiment(s) herein.

FIG. 5 illustrates a non-limiting example of the gantry motion sensing system assembly mounted in the gantry, in accordance with an embodiment(s) herein.

FIG. 6 schematically illustrates a non-limiting example of contents of the gantry motion sensing system, in accordance with an embodiment(s) herein.

FIG. 7 schematically illustrates a non-limiting example of the gantry motion evaluation module, in accordance with an embodiment(s) herein.

FIG. 8 graphically illustrates an example plot of the motion signals from the gantry motion sensing system for a particular axis over time processed by the gantry motion evaluation module, in accordance with an embodiment(s) herein.

FIG. 9 graphically illustrates an example plot of the processed motion signals from the gantry motion sensing system for the particular axis over a particular time period along with analysis criteria with a single threshold range, in accordance with an embodiment(s) herein.

FIG. 10 graphically illustrates an example plot of the processed motion signals from the gantry motion sensing system for the particular axis over a particular time period along with analysis criteria with a plurality of threshold ranges, in accordance with an embodiment(s) herein.

FIG. 11 graphically illustrates an example plot of the motion signals from the gantry motion sensing system for the same axis over a different time period or a different axis over the same or different time period processed by the gantry motion evaluation module, in accordance with an embodiment(s) herein.

FIG. 12 graphically illustrates an example plot of the processed motion signals from the gantry motion sensing system for the same axis over the different time period or the different axis over the same or different time period along with analysis criteria with a single threshold range, in accordance with an embodiment(s) herein.

FIG. 13 graphically illustrates an example of stability status graphical indicia presented to a user to provide a status of the attachment of the gantry to the examination room floor and/or the attachment of a component to the rotating frame after processing motion signals, in accordance with an embodiment(s) herein.

FIG. 14 illustrates a non-limiting example of a flow chart for a computer-implemented method for detecting a compromised attachment based on gantry motion during calibration scanning, in accordance with an embodiment(s) herein.

FIG. 15 illustrates a non-limiting example of a flow chart for a computer-implemented method for detecting a compromised attachment based on gantry motion during diagnostic scanning, in accordance with an embodiment(s) herein.

FIG. 16 illustrates a non-limiting example of a flow chart for a computer-implemented method for detecting a compromised attachment based on gantry motion during calibration scanning and diagnostic scanning, in accordance with an embodiment(s) herein.

### DETAILED DESCRIPTION

Embodiments of the present disclosure will now be described, by way of example, with reference to the figures, in which a system, a method and/or instructions on a computer readable medium for proactively detecting a compromised attachment of a gantry of a computed tomography (CT) imaging system to an examination room floor and/or an attachment of a component to a rotating frame in the gantry of the CT imaging system based on motion of the gantry sensed while the rotating frame is rotating, and initiating an action in response to the detection of the compromised attachment. As utilized herein, the term “compromised” encompasses an attachment that is mechanically out of specification and/or is trending towards being mechanically out of specification, and the term “proactive” encompasses detecting the compromised attachment at least before the detection of degraded image quality and/or imaging system damage, including prior to, early on, etc.

As briefly discussed above, reactively detecting (i.e., only after the detection of image quality degradation, image system damage, etc.) an attachment of the gantry to the examination room floor is compromised and/or an attachment of a component to the rotating frame is compromised may result in additional X-ray radiation dose exposure to the patient where a patient is re-scanned to meet image quality specifications (and X-ray radiation is ionizing radiation, which can damage and/or kill cells), the CT imaging system out-of-use for scanning for repairs along with additional expenses, etc. The approach described herein mitigates such circumstances through the proactive detection of a compromised attachment of the gantry of the CT imaging system to the examination room floor and/or the attachment of the component to the rotating frame in the gantry based on motion of the gantry.

As described in greater detail below, in one instance such proactive detection includes establishing baseline gantry motion for a CT imaging system known to be within manufacturer's specifications (e.g., during installation, service, etc.), establishing at least one threshold range about the baseline motion based on motion sensing component behavior, known and/or expected changes resulting from compromised attachments, etc., processing motion signals from a gantry motion sensing system of the gantry during calibration (e.g., quality control, quality assurance, etc.) scanning and/or diagnostic patient scanning, comparing output values from the processing with the at least one threshold range, and, in response to a value exceeding the at least one threshold range, initiating an action such as providing a notification, preventing scanning, controlling the rotation of the rotating frame, initiating a re-calibration, etc.

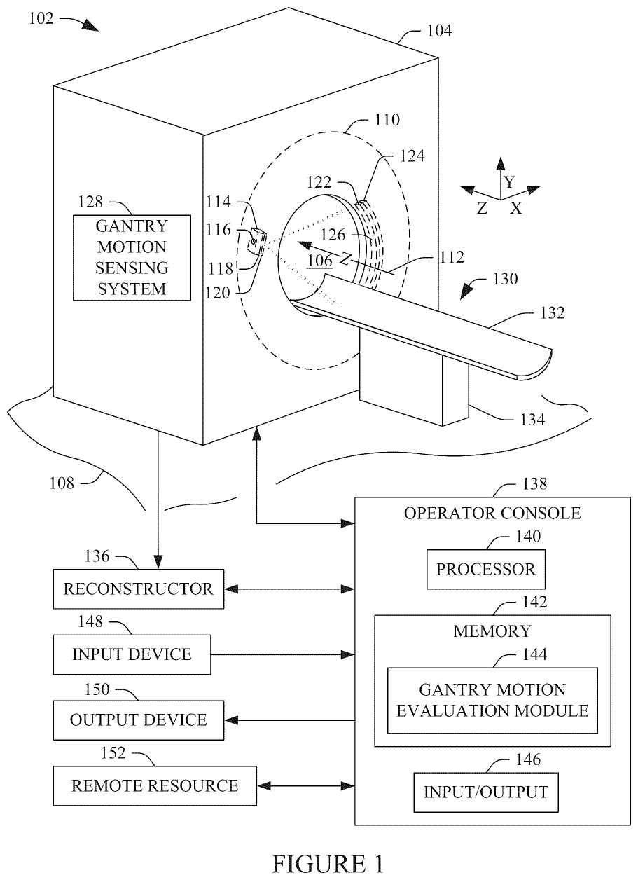

Initially referring to FIG. 1, a non-limiting example of an imaging system 102 configured for computed tomography (CT) imaging is schematically illustrated. The imaging system 102 includes a gantry 104 with a bore 106 and houses components utilized in the generation, emission and detection of X-rays, including a frame configured to rotate and a component supported thereon (some of which are described in greater detail below). The gantry 104 is mounted to a support 108, which, in this example, includes a floor of an examination room. The gantry 104 is attached to the examination room floor 108, e.g., during installation and/or service of the imaging system 102 at an entity such as a hospital, an imaging center, etc.

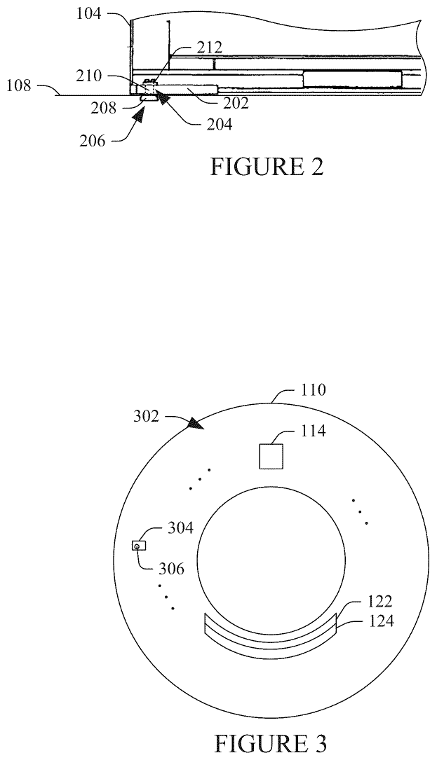

Briefly turning to FIG. 2, an example of such mounting is schematically illustrated. In this example, the gantry 104 includes a mounting bracket 202 that is configured to rest on a surface of the examination room floor 108. The mounting bracket 202 includes a mounting hole 204. A mounting element 206 such as a bolt, etc. includes a first portion 208 that is embedded and/or otherwise integrated into the examination room floor 108 and a second portion 210 that protrudes from the first portion 208 out of the examination room floor 108. The gantry 104 is positioned with respect to the examination room floor 108 to align the mounting element 206 with the mounting hole 204 such that the second portion 210 extends through the mounting hole 204. A fastener 212 such as a nut, etc. is configured to engage the second portion 210, securing the mounting bracket 202, and, hence, the gantry 104, to the examination room floor 108. The gantry 104 can include one or more such attachments.

Returning to FIG. 1, the components housed in the gantry 104 include at least a rotating frame 110. The rotating frame 110 is rotatably supported in the gantry 104, e.g., via a bearing (e.g., a slip ring, etc.) or the like, and is configured to rotate around the bore 106 about a rotational or Z-axis 112, which extends through a center of rotation (e.g., a center of the bore 106, i.e., an isocenter). In some instances, the gantry 104 is further configured to tilt. A rotating frame and gantry controller (not visible) is configured to control rotation of the rotating frame 110 and tilting of the gantry 104 when the gantry 104 is configured to tilt.

The components housed in the gantry 104 further include an X-ray source assembly 114. The X-ray source assembly 114 is supported by the rotating frame 110 and rotates in coordination with the rotating frame 110. The X-ray source assembly 114 includes an X-ray source 116 such as an X-ray tube, etc. The X-ray source 116 is configured to emit X-ray radiation having an energy at least in the X-ray diagnostic range (e.g., 20 keV to 150 keV). The X-ray assembly 114 may further include or is coupled to a filter 116 that characterizes an X-ray radiation dose profile and/or a collimator 120 that shapes the X-ray radiation to form a generally (fan, wedge, cone, etc.) shaped beam that traverses the bore 106. An X-ray controller (not visible) is configured to control components of the X-ray source assembly 114 such as X-ray radiation emission of the X-ray source 116, the collimator 120, etc.

The components housed in the gantry 104 further include a detector array 122 and a Data Acquisition System (DAS) 124. The detector array 122 and the DAS 124 are supported by the rotating frame 110, opposite the X-ray source 116 along an arc, across the bore 106, and rotate in coordination with the rotating frame 110. The detector array 122 includes a one-dimensional (1-D) or two-dimensional (2-D) array of rows of X-ray radiation sensitive detector elements 126. Each of the X-ray radiation sensitive detector elements 126 is in electrical communication with the DAS 124. The X-ray radiation sensitive detector elements 126 include an indirect conversion detector such as a scintillator/photodiode detector and/or a direct conversion detector such as a Cadmium Telluride (CdTe), a Cadmium Zinc Telluride (CZT), etc. detector. A DAS controller (not visible) controls the X-ray radiation sensitive detector array 122.

Briefly turning to FIG. 3, an example of the rotating frame 110 is schematically illustrated. The example rotating frame 110 is configured to support one or more components 302. Examples of the one or more components 302 include, but are not limited to, the X-ray source assembly 114 (FIG. 1), the detection array 122 (FIG. 1), the DAS 124 (FIG. 1), a high voltage generation system, a cooling system, balancing weights, etc. At least one of the one or more components 302 are mounted, attached, affixed, coupled, etc. to the rotating frame 110 via at least one fastener such as a nut/bolt pair, a screw, a rivet, a weld, etc. By way of non-limiting example, a component 304 of the one or more components 302 is attached to the rotating frame 110 via a fastener 306.

Returning to FIG. 1, the gantry 104 further includes a gantry motion sensing system 128. The gantry motion sensing system 128 is configured to sense certain motion of the gantry 104, including gantry motion in a X-direction and/or a Z-direction, such as motion related to the attachment of the gantry 104 to the examination room floor 108 via the mounting bracket 202 (FIG. 2) when the rotating frame 110 is rotating and/or motion related to the attachment of the one or more components 302 (FIG. 3) supported by the rotating frame 110 when the rotating frame 110 is rotating. In general, the gantry 104 will have certain characteristic motion when the attachments of the one or more components 302 to the rotating frame 110 and the gantry 104 to the examination room floor 108 are within the manufacturer's specifications, and other motion otherwise. The certain characteristic motion can be determined during installation and/or service where the attachments are known to the manufacturer's specifications.

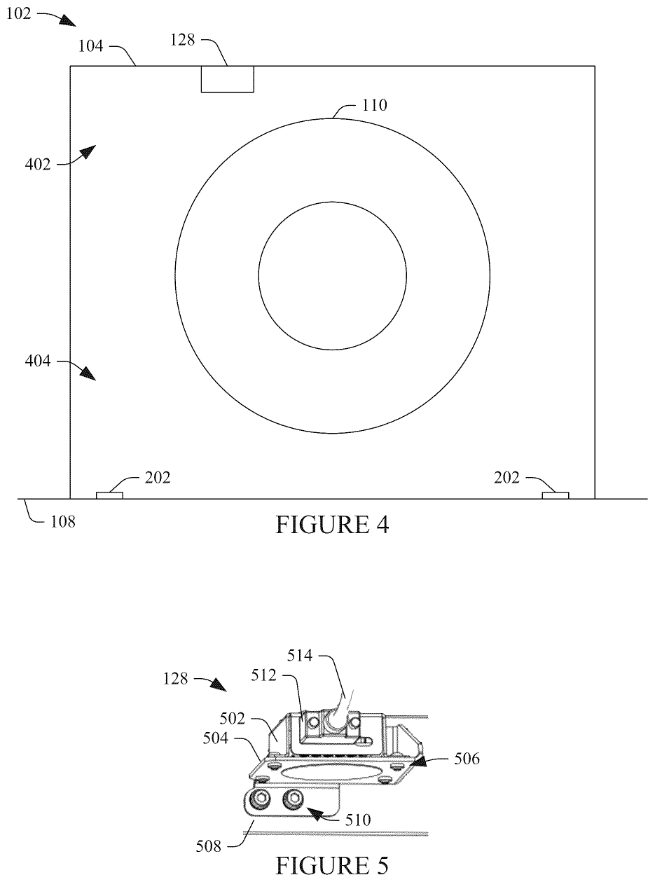

Briefly turning to FIG. 4, an example location of the gantry motion sensing system 128 within the gantry 104 is schematically illustrated. In this example, the gantry motion sensing system 128 is at a region nearer a top 402 of the gantry 104 and nearer to the rotating frame 110, where nearer the bottom 404 of the gantry 104 includes the mounting bracket 202 and the top 402 of the gantry 104 is opposite the bottom 404, relative to the examination room floor 108. However, the gantry motion sensing system 128 can be otherwise located in the gantry 104. In general, a location in the gantry 104 nearer the top 402 will experience a greater degree of motion relative to a location nearer the bottom 404, e.g., since the gantry/floor interface acts as a deflection point, and a location nearer to the rotating frame 110 facilitates coupling X and/or Z motion of the rotating frame 110 to the gantry motion sensing system 128.

Briefly turning to FIG. 5, an example structural configuration of the gantry motion sensing system 128 mounted within the gantry 104 is illustrated. In this example, the gantry motion sensing system 128 is housed in a container 502, the container 502 is coupled to a support bracket 504 via fasteners 506 (e.g., screws, nuts and bolts, rivets, a weld, etc.), and the support bracket 504 is coupled to an interior region 508 of the gantry 104 via fasteners 510 (e.g., screws, nuts and bolts, rivets, a weld, etc. An electro-mechanical connector 512 of a cable 514 is engaged with a complementary electro-mechanical connector of the gantry motion sensing system 128 (e.g., plug and socket complementary interfaces, etc.). In one instance, the cable 514 routes the motion signals off the gantry motion sensing system 128.

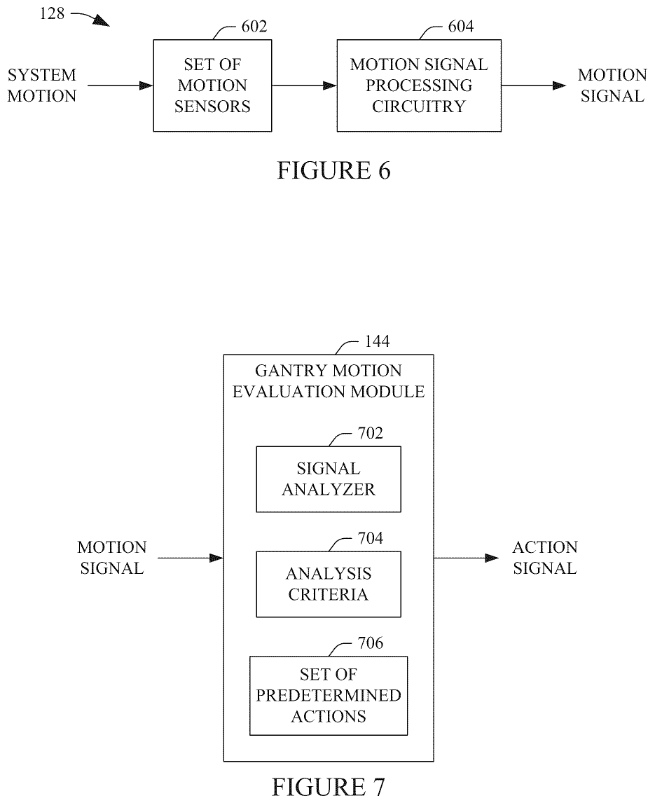

Briefly turning to FIG. 6, an example of a block diagram of the gantry motion sensing system 128 is schematically illustrated. The gantry motion sensing system 128 includes a set of one or more motion sensors 602 and motion signal processing circuitry 604. In one instance, the set of one or more motion sensors 602 and the motion signal processing circuitry 604 are located on different substrates. In another instance, the set of one or more motion sensors 602 and the motion signal processing circuitry 604 are located on a same substrates. In yet another instance, the set of one or more motion sensors 602 and the motion signal processing circuitry 604 are located in different parts of the imaging system 102. In still another instance, the motion signal processing circuitry 604 is partly or entirely located outside of the gantry 104.

Returning to FIG. 1, a table 130 includes a cradle 132 moveably coupled to a frame/base 134. In one instance, the cradle 132 is slidably coupled to the frame/base 134 via a bearing or the like, and a drive system (not visible) including a motor, a lead screw, and a nut (or other drive system) translates the cradle 132 along the frame/base 134 into and out of the bore 106 for horizontal motion, and the frame/base 134 includes a drive system (not visible) including a mechanism for vertical or diagonal motion. The cradle 132 is configured to support a subject in the bore 106 for loading, scanning, and/or unloading. A table controller (not visible) controls the drive system.

For a helical scan, the rotating frame 110 rotates in coordination with the tabletop 130 moving along the Z-axis 112, and active X-ray radiation sensitive detector elements 126 of the detector array 122 detect X-ray radiation over consecutive arc segments (integration periods) with each revolution and generate respective signals. For an axial (step and shoot) scan, the cradle 132 is positioned at a static position for each integration period and moves between integration periods. For each arc segment, the DAS 124 processes each signal and generates projection data.

A reconstructor 136 reconstructs the projection data and generates volumetric (3-D) image data for a helical scan and/or individual axial (2-D) image for an axial step and shoot scan (which can be used in combination to generate volumetric image data). The volumetric image data and/or 2-D slices thereof, and/or the individual axial images can be visually presented, filmed, etc. Examples of suitable reconstruction algorithms include filtered back projection (FBP), advanced statistical iterative reconstruction (ASIR), conjugate gradient (CG), maximum likelihood expectation maximization (MLEM), model-based iterative reconstruction (MBIR), and/or other reconstruction algorithm.

A computing system 138 serves as an “OPERATOR CONSOLE” of the imaging system 102. The computing system 138 may be a computer, a workstation, server, etc. The computing system 138 includes a processor 140 such as a microprocessor (mP), a central processing unit (CPU), graphics processing unit (GPU), etc., and a computer readable medium 142 (“MEMORY”), which includes non-transitory medium and excludes transitory medium (signals, carrier waves, and the like). The computer readable medium/memory 142 at least includes a gantry motion evaluation module 144.

In one instance the gantry motion evaluation module 144 is configured to process motion signals generated by the gantry motion sensing system 128 while the rotating gantry 110 is rotating and proactively detect a compromised attachment of the gantry 104 to the examination room floor 108 and/or an attachment of a component of the one or more components 302 (FIG. 3) to the rotating frame 110 in the gantry 104 and initiating an action in response to the detection of the compromised attachment.

As described in greater detail below, such proactive detection includes establishing baseline gantry motion for the imaging system 102 when the motion of the imaging system 102 is within manufacturer's specifications, establishing at least one threshold range about the baseline motion, processing the motion signals from the gantry motion sensing system 128 while the rotating frame 110 is rotating, comparing the processed motion signals with the at least one threshold range, and initiating an action based on the comparison.

As briefly discussed above, reactively detecting (i.e., only after the detection of image quality degradation, image system damage, etc.) an attachment of the gantry 104 to the examination room floor 108 is compromised and/or of a component to the rotating frame 110 is compromised may result in a re-scan of a patient and additional X-ray radiation dose exposure to the patient, imaging system down-time, etc. The approach described herein mitigates such circumstances through the proactive detection.

The computing system 138 further includes input/output (I/O) 146. The computing system 138 is in electrical communication with the reconstructor 136 through the I/O 146 and/or otherwise. An input device 148 includes a keyboard, mouse, touchscreen, microphone, etc. The input device 148 is in electrical communication with the computing system 138 through the I/O 146 and/or otherwise. An output device 150 includes a human readable device such as a display monitor or the like. The output device 150 is in electrical communication with the computing system 138 through the I/O 146 and/or otherwise.

A remote resource 152 includes one or more of a server, a workstation, a Radiology Information System (RIS), a Hospital Information System (HIS), an Electronic Medical Record (EMR), a PACS for storing information, a PACS further configured with image viewing and/or manipulating software, cloud resources with shared remote data storage and/or computing power including resources distributed over data centers, etc. The computing system 138 and the remote resource 152 are in communication via wired and/or wireless technologies. Such communication can be through Digital Imaging and Communications in Medicine (DICOM), Health Level Seven (HL7), etc. formats and protocols.

Moving to FIG. 7, an example of the gantry motion evaluation module 144 is schematically illustrated. The example gantry motion evaluation module 144 includes a signal analyzer 702, analysis criteria 704, and a set of predetermined actions 706. The gantry motion evaluation module 144 receives, as input, the motion signal from the gantry motion sensing system 128, and outputs an action signal. Where no action is triggered, the action may be no action, or no action signal is output. The signal analyzer 702 is configured to analyze the received motion signal based on the analysis criteria 704.

To determine the analysis criteria 704, in one instance, the imaging system 102 is operated with the rotating frame 110 rotating at different speeds, e.g., each of the scanning speeds of the rotating frame 110 for the particular configuration of the imaging system 102, a sub-set (i.e., less than all) of the speeds of the rotating frame 110 for the particular configuration of the imaging system 102, etc. to acquire baseline motion data, where the attachments of the components 302 (FIG. 3) and/or the gantry 104 (FIG. 1) are known to be within the manufacturer's specifications. The analysis criteria 704 can then be established based on the baseline data, accounting for sensor noise, repeatability, design margin, moving parts (e.g., the filter 118, the collimator 120, etc.), circuit board variations (e.g., gain, etc.), estimated changes in motion due to a compromised attachment (e.g., from previous occurrences, a model, etc.), etc.

For the analysis, the signal analyzer 702 receives the motion signals from the gantry motion sensing system 128 when the rotating frame 110 reaches a steady state rotation speed. In one instance, the signal analyzer 702 receives motion signals every time the rotating frame 110 reaches a steady state rotation speed (e.g., 120 rpm±tolerance for a 120 rpm scan, etc.), whether during installation, service, calibration, warm up, patient scanning, etc. In another instance, the signal analyzer 702 receives the motion signals less than every time the rotating frame 110 reaches a steady state rotation speed, e.g., only during calibration, only during patient scanning, based on the time of day, based on the day of the week, based on a predetermined number of scans since the previous time the motion signals were received, etc.

In one instance, the signal analyzer 702 analyzes motion signals as they are received. In another instance, the signal analyzer 702 analyzes the motion signals only after a certain set of motion signals are received, e.g., after a patient scan, after a calibration, etc. In another instance, the motion signals are stored in buffer memory, and the signal analyzer 702 analyzes the stored motion signals only after certain criteria is met (e.g., a given number of rotations of the rotating frame 110, lapse of a given time period, etc.). In another instance, the signal analyzer 702 analyzes the motion signals on-demand, e.g., based on a user input. In another instance, the signal analyzer 702 analyzes the motion signals before rotating the rotating gantry 110.

The analysis, in one instance, includes comparing processed motion signals to the analysis criteria 704, such as to one or more predetermined threshold ranges. In one instance, a result of the comparison determines a state of the attachment of the gantry 104 to the examination room floor 108 and/or a component of the components 302 to the rotating frame 110. In one instance, where the one or more predetermined threshold ranges is a single threshold range, the state is either the attachment is within a healthy system threshold range or not. Where the one or more predetermined threshold ranges includes multiple threshold ranges, the state outside of the system threshold range will include multiple states or sub-states.

The signal analyzer 702, based on the result of the analysis, performs an action of the set of predetermined actions 706 immediately or near immediately (e.g., within a given time limit, etc.) after failing the analysis criteria 704. The time limit may be determined by which threshold ranges are exceeded by the processed motion signals, etc. Examples of actions include providing a notification such as a notification to the user and/or service provider, etc. For example, in one instance the gantry motion evaluation module 144 provides a message on the display monitor of the operator console 138 to notify the user of a potential issue and/or possible effect of the issue. Additionally, or alternatively, the gantry motion evaluation module 144 provides an identification of the issue to service personnel to start a process to deploy a service person to the site to evaluate the imaging system.

Further examples of actions include limiting a speed of the rotating frame 110, preventing the rotating frame 110 from rotating, stopping the rotating frame 110 from rotating, etc. For example, in one instance the gantry motion evaluation module 144 limits the speed of the rotating frame 110 where a slower rotation speed may be allowable due to the overall lower potential for gantry motion, whereas a faster rotation speed may have more potential for imbalance to move the gantry and/or experience resonances. In another instance, the gantry motion evaluation module 144 controllably ramps the rotating frame 110 down to a stopped position. In another instance, the gantry motion evaluation module 144 prohibits ramping up and rotating the rotating frame 110 when the rotating frame 110 is currently not rotating.

Further examples of actions include initiating a re-calibration. For example, in certain situations, the detected issue may be able to be corrected or calibrated out. An example includes, but is not limited to, a spatial shift in one or more of the components 302 on the rotating frame 110 such as a shift of the X-ray tube 116, the filter 118, the collimator 120, and/or the detector array 122 (FIG. 1). A predictable shift could be calibrated out through a re-calibration. The above-noted examples of suitable actions are not limiting, and other actions are contemplated herein.

Additionally, or alternatively, the processed motion signals are trended over time. For example, multiple sets of acquired motion signals corresponding to different calibration scanning events and/or different diagnostic scanning events can be accumulated and evaluated in combination. In one instance, the gantry motion evaluation module 144 is triggered to perform an action even when the processed motion signal falls within a healthy system threshold range, e.g., when the trending indicates the processed motion signals are trending towards falling outside of the healthy system threshold range.

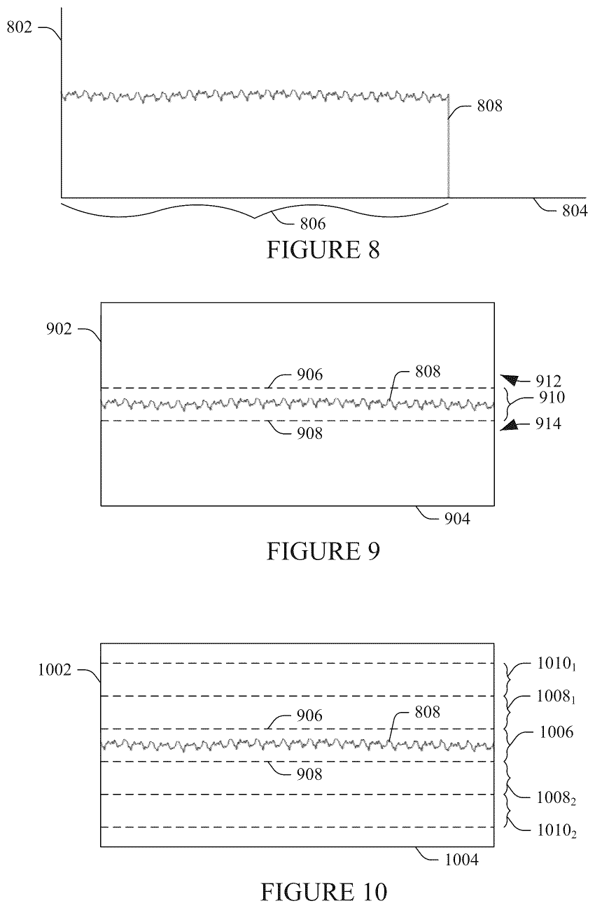

As described herein, the gantry motion signal evaluation module 144 processes motion signals from the gantry motion sensing system 128. FIG. 8 graphically illustrates example results of such processing for a particular axis (e.g., X or Z). In FIG. 8, a first axis 802 represents an amplitude of motion signals along the axis. A second axis 804 represents time. In this example, motion signals are processed over a given time period 806. A plot 808 of the processed motion signals represents back and forth motion of the gantry 104 along the axis.

FIG. 9 graphically illustrates a comparison of the processed signal described in connection with FIG. 8 (i.e., the plot 808) against the analysis criteria 704. Similarly, a first axis 902 represents an amplitude of motion signals along a particular axis and a second axis 904 represents time. FIG. 9 further shows analysis thresholds, including a first threshold 906 for a first direction along the axis, and a second threshold 908 for an opposing direction along the axis, together defining threshold ranges 910, 912 and 914. In FIG. 9, an entirety of the plot 808 is within the first and second thresholds 906 and 908 (i.e., the threshold range 910) and would not trigger an action from the gantry motion signal evaluation module 144.

FIG. 10 graphically illustrates another comparison of the processed signal described in connection with FIG. 8 (i.e., the plot 808) against different the analysis criteria 704. Similarly, a first axis 1002 represents an amplitude of motion signals along a particular axis and a second axis 1004 represents time. In FIG. 10, the analysis criteria includes a set of threshold ranges, including a first range 1006 (the range 910 for the thresholds 906 and 908 of FIG. 9), . . . Ith ranges 1008₁ and 1008₂, . . . , and an Nth ranges 1010₁ and 1010₂, where I and N are positive integers. In this example, the ranges 1006, 1008₁ and 1008₂, and 1010₁ and 1010₂ map to different sets of actions.

For example, in one instance the first range 1006, as discussed in connection with FIG. 9, does not trigger an action from the gantry motion signal evaluation module 144, while the Ith ranges 1008₁ and 1008₂ trigger the gantry motion signal evaluation module 144 to transmit a notification, and the Nth ranges 1010₁ and 1010₂ may trigger the gantry motion signal evaluation module 144 to additionally control the imaging system 102, e.g., prevent scanning, control the rotational speed of the rotating frame 110, etc. In FIG. 10, N=6 with ranges 1008₁ and 1008₂ represent a group and ranges 1010₁ and 1010₂ represent another group. In other examples, N is greater or smaller. In addition, the ranges can be of similar or different sizes.

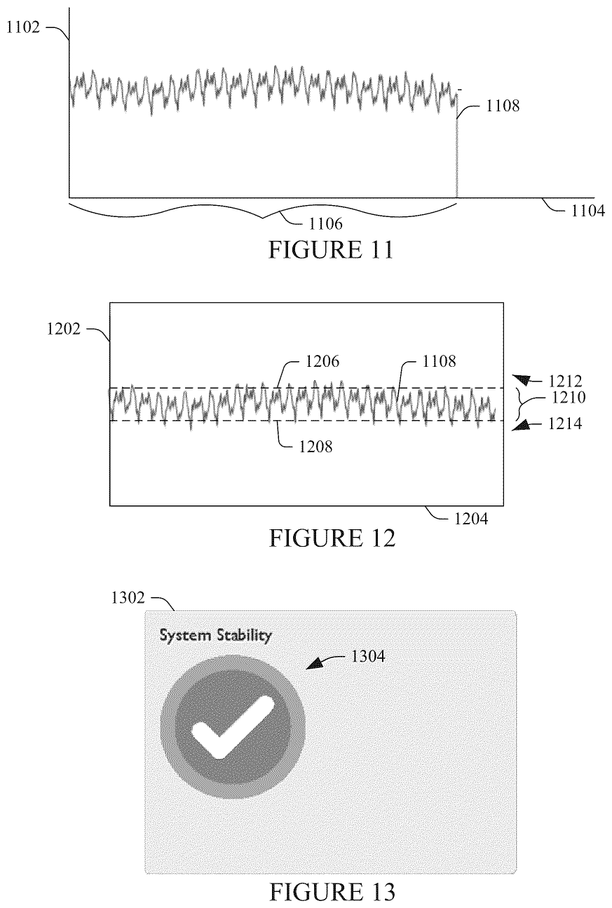

FIG. 11 graphically illustrates example results of processing for the same axis (e.g., X or Z) as in FIG. 8 at a different time or a different axis (e.g., Z or X) at the same time or a different time as in FIG. 8. In FIG. 11, a first axis 1102 represents an amplitude of motion signals along the axis. A second axis 1104 represents time. In this example, motion signals are processed over a given time period 1106. A plot 1108 of the processed motion signals represents back and forth motion of the gantry 104 along the axis. For explanatory purposes, the peak amplitudes in the plot 1108 are greater than the peak amplitudes in the plot 808.

FIG. 12 graphically illustrates a comparison of the processed signal described in connection with FIG. 11 (i.e., the plot 1108) against the analysis criteria 704. Similarly, a first axis 1202 represents an amplitude of motion signals along a particular axis and a second axis 1204 represents time. FIG. 12 further shows analysis thresholds, including a first threshold 1206 for a first direction along the axis, and a second threshold 1208 for an opposing direction along the axis, together defining threshold ranges 1210, 1212 and 1214. In FIG. 12, some of the peaks (maximums and minimums) the plot 1108 exceeds the first and second thresholds 1206 and 1208 (i.e., are outside of the threshold range 1210 and in either the threshold range 1212 or 1214), and would trigger an action from the gantry motion signal evaluation module 144.

With reference to FIGS. 1, 9, 10 and 12, regardless of which of the threshold ranges peaks of the processed motion signals (e.g., the plot 808, the plot 1108, etc.) are in, the gantry motion evaluation module 144 provides a system stability status. Briefly turning to FIG. 13, example system stability status graphical indicia 1302 provides a status of the system stability. In this example, the graphical indicia 1302 includes a “check” 1304 for system stability, indicating no mechanical compromise was detected with the attachment of the gantry 104 to the examination room floor 108 and/or a component of the components 302 to the rotating frame 110. Indicia other than a “check” is contemplated herein. For example, color, patterns, shapes, sizes, etc. are contemplated herein. In addition, other graphical indicia (e.g., an orange or yellow triangle, a red stop sign, etc.) is utilized when it is determined the attachment of the gantry 104 to the examination room floor 108 and/or a component of the components 302 to the rotating frame 110 is compromised.

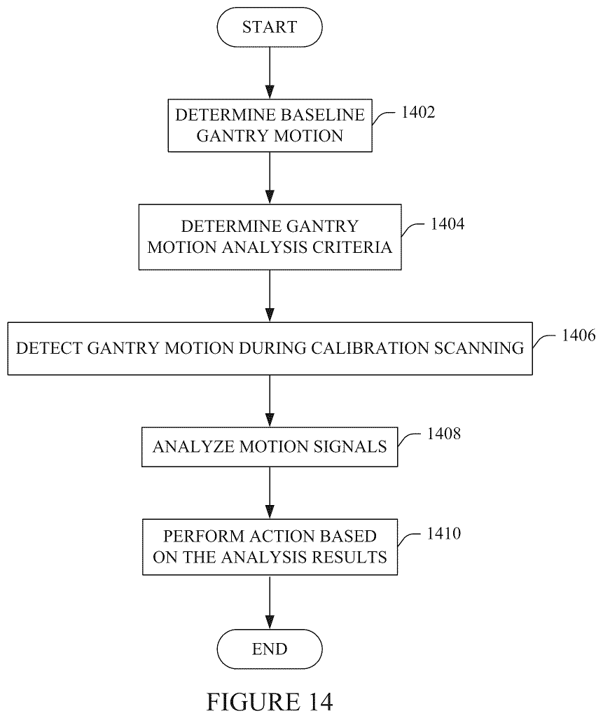

FIG. 14 illustrates a non-limiting example of a flow chart for a computer-implemented method for detecting a compromised attachment based on gantry motion during calibration scanning, in accordance with an aspect herein. It is to be appreciated that the ordering of the acts in the method is not limiting. As such, other orderings are contemplated herein. In addition, one or more acts may be omitted, and/or one or more additional acts may be included.

At 1402, baseline gantry motion is determined, as described herein and/or otherwise. For example, during installation, the imaging system 102 is operated with the rotating frame 110 rotating at the different rotating gantry rotational speeds of the imaging system 102, and baseline motion data is acquired, where the attachments of the components 302 and/or gantry 104 are known to be within the manufacturer's specifications.

At 1404, gantry motion analysis criteria 704 is determined, as described herein and/or otherwise. In one instance, the analysis criteria 704 is established based on the baseline data, accounting for sensor noise, repeatability, design margin, moving parts, circuit board variations, estimated changes in motion due to a compromised attachment (e.g., from previous occurrences, a model, etc.), etc. In one instance, the analysis criteria 704 include one or more threshold ranges. Other approaches are also contemplated herein.

At 1406, gantry motion is detected during calibration scanning, as described herein and/or otherwise. For example, in one instance the gantry motion sensing system 128 senses gantry motion in a X-direction and/or a Z-direction related to the attachment of the gantry 104 to the examination room floor 108 and/or a component 302 to the rotating frame 110 during calibration scanning and outputs motion signals indicative thereof. Such gantry motion can be detected daily before the first diagnostic scan of a patient, or less or more frequently.

At 1408, the motion signals are analyzed, as described herein and/or otherwise. For example, in one instance the signal analyzer 702 processes the motion signals and compares them to the analysis criteria 704, as described herein and/or otherwise. For example, in one instance the signal analyzer 702 compares the processed motion signal to one or more predetermined threshold ranges to determine a state of the attachment of the gantry 104 to the examination room floor 108 and/or a component of the components 302 to the rotating frame 110.

At 1410, the gantry motion evaluation module 144 performs an action based on a result of the comparison, as described herein and/or otherwise. For example, where the processed motion signal falls within a healthy system threshold range, the action is no action and/or update displayed graphical system stability indicia 1302. However, where the processed motion signal is outside of the healthy system threshold range, the gantry motion evaluation module 144 performs an action such as transmit notification, prevent scanning, control rotation of the rotating frame 110, initiate a re-calibration, etc.

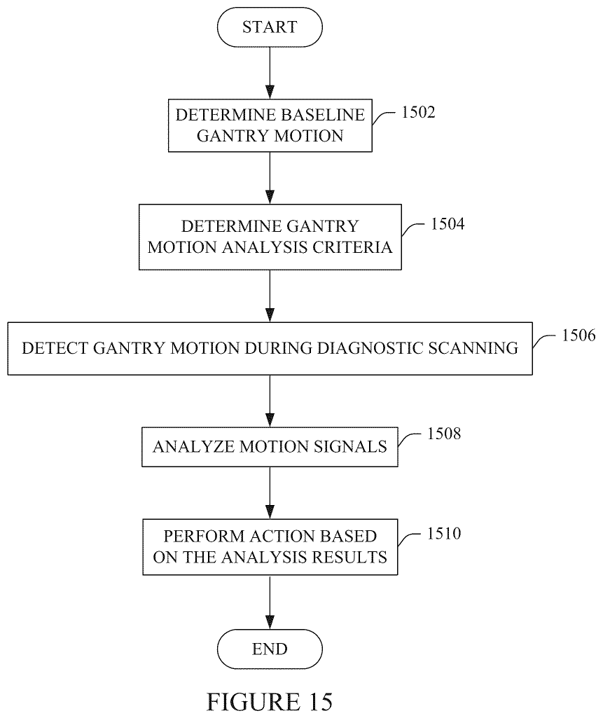

FIG. 15 illustrates a non-limiting example of a flow chart for a computer-implemented method for detecting a compromised attachment based on gantry motion during diagnostic scanning, in accordance with an aspect herein. It is to be appreciated that the ordering of the acts in the method is not limiting. As such, other orderings are contemplated herein. In addition, one or more acts may be omitted, and/or one or more additional acts may be included.

At 1502, baseline gantry motion is determined, as described herein and/or otherwise. For example, during installation, the imaging system 102 is operated with the rotating frame 110 rotating at the different rotating gantry rotational speeds of the imaging system 102, and baseline motion data is acquired, where the attachments of the components 302 and/or gantry 104 are known to be within the manufacturer's specifications.

At 1504, gantry motion analysis criteria 704 is determined, as described herein and/or otherwise. In one instance, the analysis criteria 704 is established based on the baseline data, accounting for sensor noise, repeatability, design margin, moving parts, circuit board variations, estimated changes in motion due to a compromised attachment (e.g., from previous occurrences, a model, etc.), etc. In one instance, the analysis criteria 704 include one or more threshold ranges. Other approaches are also contemplated herein.

At 1506, gantry motion is detected during diagnostic scanning, as described herein and/or otherwise. For example, in one instance the gantry motion sensing system 128 senses gantry motion in a X-direction and/or a Z-direction related to the attachment of the gantry 104 to the examination room floor 108 and/or a component 302 to the rotating frame 110 during calibration scanning and outputs motion signals indicative thereof. Such gantry motion can be detected during each patient scan, or less or more frequently.

At 1508, the motion signals are analyzed, as described herein and/or otherwise. For example, in one instance the signal analyzer 702 processes the motion signals and compares them to the analysis criteria 704, as described herein and/or otherwise. For example, in one instance the signal analyzer 702 compares the processed motion signal to one or more predetermined threshold ranges to determine a state of the attachment of the gantry 104 to the examination room floor 108 and/or a component of the components 302 to the rotating frame 110, depending on which threshold ranges have been crossed.

At 1510, the gantry motion evaluation module 144 performs an action based on a result of the comparison, as described herein and/or otherwise. For example, where the processed motion signal falls within a healthy system threshold range, the action is no action or update displayed graphical system stability indicia 1302. However, where the processed motion signal is outside of the healthy system threshold range, the gantry motion evaluation module 144 performs an action such as transmit notification, prevent scanning, control rotation of the rotating frame 110, initiate a re-calibration, etc.

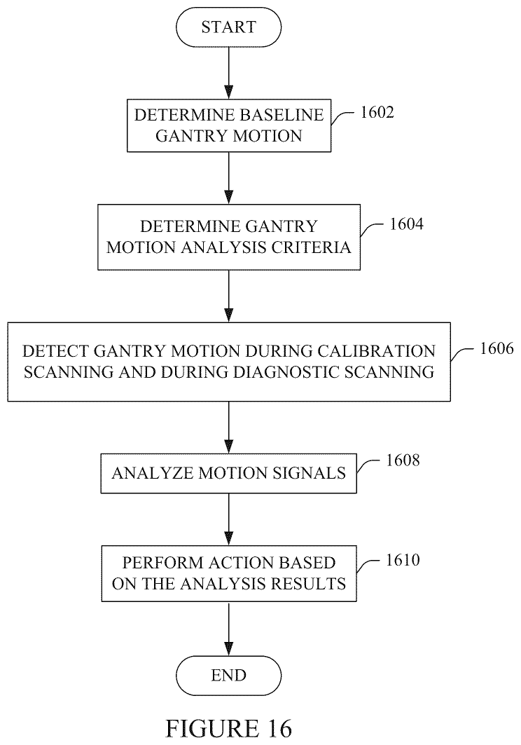

FIG. 16 illustrates a non-limiting example of a flow chart for a computer-implemented method for detecting a compromised attachment based on gantry motion during calibration scanning and diagnostic scanning, in accordance with an aspect herein. It is to be appreciated that the ordering of the acts in the method is not limiting. As such, other orderings are contemplated herein. In addition, one or more acts may be omitted, and/or one or more additional acts may be included.

At 1602, baseline gantry motion is determined, as described herein and/or otherwise. For example, during installation, the imaging system 102 is operated with the rotating frame 110 rotating at the different rotating gantry rotational speeds of the imaging system 102, and baseline motion data is acquired, where the attachments of the components 302 and/or gantry 104 are known to be within the manufacturer's specifications.

At 1604, gantry motion analysis criteria 704 is determined, as described herein and/or otherwise. In one instance, the analysis criteria 704 is established based on the baseline data, accounting for sensor noise, repeatability, design margin, moving parts, circuit board variations, estimated changes in motion due a compromised attachment (e.g., from previous occurrences, a model, etc.), etc. In one instance, the analysis criteria 704 include one or more threshold ranges. Other approaches are also contemplated herein.

At 1606, gantry motion is detected during diagnostic scanning, as described herein and/or otherwise. For example, in one instance the gantry motion sensing system 128 senses gantry motion in a X-direction and/or a Z-direction related to the attachment of the gantry 104 to the examination room floor 108 and/or a component 302 to the rotating frame 110 during calibration scanning and during patient scanning, and outputs motion signals indicative thereof. Such gantry motion can be detected during each patient scan, or less or more frequently.

At 1608, the motion signals are analyzed, as described herein and/or otherwise. For example, in one instance the signal analyzer 702 processes the motion signals and compares them to the analysis criteria 704, as described herein and/or otherwise. For example, in one instance the signal analyzer 702 compares the processed motion signal to one or more predetermined threshold ranges to determine a state of the attachment of the gantry 104 to the examination room floor 108 and/or a component of the components 302 to the rotating frame 110.

At 1610, the gantry motion evaluation module 144 performs an action based on a result of the comparison, as described herein and/or otherwise. For example, where the processed motion signal falls within a healthy system threshold range, the action is no action or update displayed graphical system stability indicia 1302. However, where the processed motion signal is outside of the healthy system threshold range, the gantry motion evaluation module 144 performs an action such as transmit notification, prevent scanning, control rotation of the rotating frame 110, initiate a re-calibration, etc., depending on which threshold ranges have been crossed.

The above can be implemented by way of computer readable instructions, encoded, or embedded on the computer readable storage medium, which, when executed by a computer processor, cause the processor to carry out the described acts or functions. Additionally, or alternatively, at least one of the computer readable instructions is carried out by a signal, carrier wave or other transitory medium, which is not computer readable storage medium.

Additionally, or alternatively, the processed motion signals are trended over time. For example, multiple sets of acquired motion signals corresponding to different calibration scanning events and/or different diagnostic scanning events can be accumulated and evaluated in combination. In one instance, the gantry motion evaluation module 144 is triggered to perform an action even when the processed motion signal falls within a healthy system threshold range, e.g., when the trending indicates the processed motion signals are trending towards falling outside of the healthy system threshold range.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include such additional elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”. The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present disclosure. That is, features of the described embodiments can be combined with any appropriate aspect described above and optional features of any one aspect can be combined with any other appropriate aspects. Similarly, features set forth in dependent claims can be combined with non-mutually exclusive features of other dependent claims, particularly where the dependent claims depend on the same independent claim. Single claim dependencies may have been used as practice in some jurisdictions that require them, but this should not be taken to mean that the features in the dependent claims are mutually exclusive.

What is claimed is:

1. A computed tomography imaging system, comprising:
a gantry mounted to a floor, the gantry including;
a rotating frame rotatably supported in the gantry;
at least one component mounted to the rotating frame; and
a gantry motion sensing system configured to sense, while the rotating frame is rotating, a motion of the gantry that is indicative of a state of one of an attachment of the gantry to the floor and an attachment of the at least one component to the rotating frame and generate a motion signal indicative of the state; and
motion signal processing circuitry configured to process the motion signal, and, in response to determining that the motion signal exceeds at least one predetermined threshold, transmit a notification that indicates at least one of the attachment of the gantry to the floor and the attachment of the at least one component to the rotating frame is compromised.

2. The computed tomography imaging system of claim 1, wherein the notification is transmitted to at least one of a user of the computed tomography imaging system and a service provider of the computed tomography imaging system.

3. The computed tomography imaging system of claim 1, wherein the motion signal processing circuitry is further configured to prevent scanning in response to determining that the motion signal exceeds the at least one predetermined threshold.

4. The computed tomography imaging system of claim 1, wherein the motion signal processing circuitry is further configured to one of prevent the rotating frame from rotating, limit rotational speeds of the rotating frame, and stop the rotating frame from rotating in response to determining that the motion signal exceeds the at least one predetermined threshold.

5. The computed tomography imaging system of claim 1, further comprising:
an operator console with a display monitor, wherein the motion signal processing circuitry is further configured to display graphical indicia that indicates the state via the display monitor.

6. The computed tomography imaging system of claim 1, wherein the motion signal processing circuitry is further configured to initiate a re-calibration in response to determining that the motion signal exceeds the at least one predetermined threshold.

7. The computed tomography imaging system of claim 1, wherein the at least one predetermined threshold includes a plurality of threshold ranges, each associated with a different set of actions.

8. The computed tomography imaging system of claim 1, wherein the gantry motion sensing system is configured to sense the motion of the gantry during calibration scanning.

9. The computed tomography imaging system of claim 1, wherein the gantry motion sensing system is configured to sense the motion of the gantry during diagnostic scanning.

10. The computed tomography imaging system of claim 1, wherein the at least one predetermined threshold is determined based at least on baseline motion data acquired during a motion state of the computed tomography imaging system within manufacturer's specifications.

11. A computer-implemented method, comprising:
rotating a rotating frame of a gantry of a computed tomography imaging system, wherein the gantry is mounted to a floor and at least one component is mounted to the rotating frame;
sensing a motion of the gantry that is indicative of a state of one of an attachment of the gantry to the floor and an attachment of the at least one component to the rotating frame and generate a motion signal indicative of the state; and
transmitting, in response to determining that the motion signal exceeds at least one predetermined threshold, a notification that indicates at least one of the attachment of the gantry to the floor and the attachment of the at least one component to the rotating frame is compromised.

12. The computer-implemented method of claim 11, wherein the notification is transmitted to at least one of a user of the computed tomography imaging system and a service provider of the computed tomography imaging system.

13. The computer-implemented method of claim 11, further comprising:
preventing scanning in response to determining that the motion signal exceeds the at least one predetermined threshold.

14. The computer-implemented method of claim 11, further comprising:
one of preventing the rotating frame from rotating, limiting rotational speeds of the rotating frame, and stopping the rotating frame from rotating in response to determining that the motion signal exceeds the at least one predetermined threshold.

15. The computer-implemented method of claim 11, further including:
visually presenting graphical indicia that indicates the state on a display monitor of the computed tomography imaging system.

16. A computer readable medium encoded with computer executable instructions, which, when executed by a processor, causes the processor to:
rotate a rotating frame of a gantry of a computed tomography imaging system, wherein the gantry is mounted to a floor and at least one component is mounted to the rotating frame;
sense a motion of the gantry that is indicative of a state of one of an attachment of the gantry to the floor and an attachment of the at least one component to the rotating frame and generate a motion signal indicative of the state; and
transmit, in response to determining that the motion signal exceeds at least one predetermined threshold, a notification that indicates at least one of the attachment of the gantry to the floor and the attachment of the at least one component to the rotating frame is compromised.

17. The computer readable medium of claim 16, wherein the notification is transmitted to at least one of a user of the computed tomography imaging system and a service provider of the computed tomography imaging system.

18. The computer readable medium of claim 17, wherein the computer executable instructions further cause the processor to:
prevent scanning in response to determining that the motion signal exceeds the at least one predetermined threshold.

19. The computer readable medium of claim 18, wherein the computer executable instructions further cause the processor to:
one of prevent the rotating frame from rotating, limit rotational speeds of the rotating frame, and stop the rotating frame from rotating in response to determining that the motion signal exceeds the at least one predetermined threshold.

20. The computer readable medium of claim 19, wherein the computer executable instructions further cause the processor to:
visually present graphical indicia that indicates the state on a display monitor of the computed tomography imaging system.
